(19) United States
(12) Patent Application Publication
(10) Pub. No.: US 2022/0344037 A1
(43) Pub. Date: Oct. 27, 2022

(54) NURSING INFORMATION MODULE AUTOMATION SYSTEM AND METHOD

(71) Applicant: SMART AGEING TECH CO., LTD., New Taipei City (TW)

(72) Inventors: Cheng-Yang Liu, New Taipei City (TW); Shu Hao Wu, New Taipei City (TW); Shih-Chung Kang, New Taipei City (TW); Yi-Fen Li, New Taipei City (TW); Chia-Min Kao, New Taipei City (TW)

(21) Appl. No.: 17/658,482

(22) Filed: Apr. 8, 2022

(30) Foreign Application Priority Data
Apr. 26, 2021 (TW) 110114966

(51) Int. Cl. G16H 40/20; G06N 5/04

(52) U.S. Cl. CPC G16H 40/20; G06N 5/04; G06F 40/30

(57) ABSTRACT

The present invention relates to a nursing information module automation system and method. The method includes steps of detecting a nursing record inputted by a user; transmitting the nursing record to a nursing information module automation natural language processing model to perform a focus prediction, so as to automatically predict at least one nursing focus based on the nursing record; and recommending at least one recommended module in accordance with the at least one nursing focus by the nursing information module automation natural language processing model.

### CROSS-REFERENCE TO RELATED APPLICATION

The present application is based upon and claims the benefit of priority to Taiwan Invention Patent Application Number 110114966, filed on Apr. 26, 2021 in Taiwan intellectual property office, now pending, the entire disclosure of which is incorporated herein by reference, as if fully set forth herein.

### COPYRIGHT NOTICE

A portion of this patent document contains material that is subject to copyright protection. The copyright owner has no objection to the facsimile reproduction by anyone of the patent document, as it appears in the Patent and Trademark Office patent files or records, but otherwise reserves all copyright rights whatsoever.

### FIELD

The present invention relates to a nursing information module automation system and method, in particular to an operation automation system and method applied in a nursing information system (NIS) based on a natural language processing (NLP) technology.

### BACKGROUND

A nursing record is a written record of a patient's care received by a nurse, including the content of the nursing assessment, the patient's health problems or nursing diagnosis, the nursing care provided, and the patient's condition after the nursing care. A nursing record can also be deemed a continuous description of a nursing process.

In the field of nursing, common methods of producing nursing records basically include the following several kinds: a focus charting scheme, a problem-oriented recording scheme, e.g.: S.O.A.P.I.E.R. or S.O.A.P., a source-oriented systems recording scheme, a source-oriented narrative recording scheme, a process recording scheme, etc. Among these methods, the focus charting method provides a recording system that can better express nursing problems and nursing processes, and therefore is widely used in Taiwan.

The focus charting method is a systematic method of recording nursing problems and nursing processes. It focuses on the patient's most essential and primary problem, and describes the condition, illness, symptoms, or events that occurred and the nursing activities performed by the nurse in response, and also the patient's response after receiving care. The method writes the above contents in concise, organized, systematic, and meaningful terms into nursing records. Specifically, the method keeps records in the following four aspects: Date, Action, Response, and Teaching, and therefore the focus charging method is called the DART method as well.

However, in the clinical practice of nursing, nurses are required to produce more than just nursing records. Many items need to be additionally recorded in corresponding files, including inter- or intra-hospital transfers, abnormal events, infectious diseases, and assessment-related quality indicators, such as falls, infections, pressure injuries, restraints, pain, weight changes, and other special events. Practically, nurses may only pay attention to writing nursing records for forgetting to fill in the files, being busy or inexperienced, and therefore may neglect to fill in corresponding files for special events, resulting in incomplete records, underestimation of abnormal events, and inaccurate analysis of quality indicators.

Therefore, nursing information systems (NIS) have been introduced to assist nurses in their daily nursing work with information systems by digitizing documentation. Although NIS reduces the hassle of transcribing and storing large amounts of documents, the operation is still very tedious and complicated, causing a burden to nurses. For example, operators are required to remember the location of each module in the information system, and have to enter information repeatedly if two or more modules are requiring for inputting the same information. Therefore, there is an urgent need to propose a digital tool that can automate the operation of NIS as much as possible to assist nurses in making nursing records. By using module recommendation systems to fill out relevant professional modules quickly, the efficiency of filling out the modules could be increased, and the data integrity of professional modules could be ensured as well.

Hence, there is a need to solve the above deficiencies/issues.

### SUMMARY

In view of the inadequacy of the conventional technology, the nursing information module automation system and method proposed in the present invention could interpret nursing records and recommend care modules, so that the caregiver could record the care process according to the status of the patient, and could get assistance in searching for relevant modules which can be then recommended to the caregiver to improve their work efficiency. The system provided in the present invention can also assist institutional nurses in searching for unfilled modules related to the content of nursing records, and the process could be combined with semantics extraction technology to improve the efficiency of filling out the modules. In addition, the system could also assist institutional caregivers by bringing in the content related to the daily care modules if there are any saved in the nursing records. Furthermore, the system could assist professional physicians by providing professional modules such as nutrition and functional/physical therapy assessment. Modules are recommended through reminding mechanisms based on care focus predictions. We hope to provide a more comprehensive and more convenient system for caretakers in every position.

Accordingly, the present invention provides a nursing information module automation method. The method includes detecting a nursing record inputted by a user; transmitting the nursing record to a nursing information module automation natural language processing model to perform a focus prediction, so as to automatically predict at least one nursing focus based on the nursing record; and recommending at least one recommended module for the user automatically in accordance with the at least one nursing focus by the nursing information module automation natural language processing model.

Preferably, the nursing information module automation method further includes one of following steps: accessing a focus module knowledge database by the nursing information module automation natural language processing model, to recommend the at least one recommended module for the user, in accordance with a focus and module matching relationship in the focus module knowledge database based on the at least one nursing focus; and implementing a semantics extraction technology by the nursing information module automation natural language processing model, to import the nursing record inputted by the user into the at least one recommended module automatically.

Preferably, the nursing information module automation method further includes one of following steps: implementing a word embedding technology for an unlabeled corpus and a labeled text to transform a word into a numerical status; pre-training the nursing information module automation natural language processing model based on the unlabeled corpus by using an unsupervised machine learning scheme; fine-tuning the nursing information module automation natural language processing model based on the labeled text by using a supervised machine learning scheme.

Preferably, the word embedding technology is selected from one of a One-hot encoding technology, a Word2Vec technology, a Doc2Vec technology, a Glove technology, a FastText technology, an ELMO technology, a GPT technology, a BERT technology and a combination thereof.

Preferably, the nursing information module automation natural language processing model is selected from one of a Transformer model, a BERT model, an ELMO model, a LSTM model, a GPT1.0 model, a GPT2.0 model, a Flair model, a StanfordNLP model, a ULMFiT model and a combination thereof.

Accordingly, the present invention further provides a nursing information module automation system. The system includes a system server configured to install an intelligent nursing platform including the nursing information module automation natural language processing model; and a user equipment communicatively connected with the and system server and configured to install a frontend interface program to detect a nursing record inputted by a user, wherein the user equipment transmits the nursing record to the nursing information module automation natural language processing model to perform a focus prediction, so as to automatically predict at least one nursing focus based on the nursing record, and generate at least one recommended module based on the predicated at least one nursing focus, and displays the at least one recommended module for the user via the frontend interface program.

Preferably, the intelligent nursing platform further includes one of a nursing record module, a tubing record module, a resident variation module, a daily care module, and the at least one recommended module.

Preferably, the user inputs the nursing record into the intelligent nursing platform by operating the nursing record module.

Preferably, by implementing a semantics extraction technology, the nursing information module automation natural language processing model imports the nursing record inputted by the user by operating the nursing record module into the at least one recommended module automatically.

Preferably, the user equipment is selected from one of a mobile device, a smart phone, a tablet device, a desktop computer and a notebook computer.

### DESCRIPTION OF THE DRAWINGS

A more complete appreciation of the invention and many of the attendant advantages thereof are readily obtained as the same become better understood by reference to the following detailed description when considered in connection with the accompanying drawing, wherein:

FIG. 1 is a structural schematic view illustrating a nursing information module automation system in accordance with the present invention;

FIG. 2 is a schematic view illustrating the function modules contained in a fully functional intelligent nursing platform provided to caretakers through the front-end program in accordance with the present invention;

FIG. 3 is a flow chart illustrating operational steps for the nursing information module automation natural language processing model provided in accordance with the present invention;

FIG. 4 and FIG. 5 are schematic views illustrating the operations of the nursing information module automation natural language processing model while it is running in a functional module operation interface provided in accordance with the present invention; and

FIG. 6 is a schematic view illustrating how the nursing information module automation system operates in a webpage browser provided in accordance with the present invention.

### DETAILED DESCRIPTION

The present disclosure will be described with respect to particular embodiments and with reference to certain drawings, but the disclosure is not limited thereto but is only limited by the claims. The drawings described are only schematic and are non-limiting. In the drawings, the size of some of the elements may be exaggerated and not drawn on scale for illustrative purposes. The dimensions and the relative dimensions do not necessarily correspond to actual reductions to practice.

It is to be noticed that the terms “comprising” and “including”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device including means A and B” should not be limited to devices consisting only of components A and B.

The disclosure will now be described by a detailed description of several embodiments. It is clear that other embodiments can be configured according to the knowledge of persons skilled in the art without departing from the true technical teaching of the present disclosure, the claimed disclosure being limited only by the terms of the appended claims.

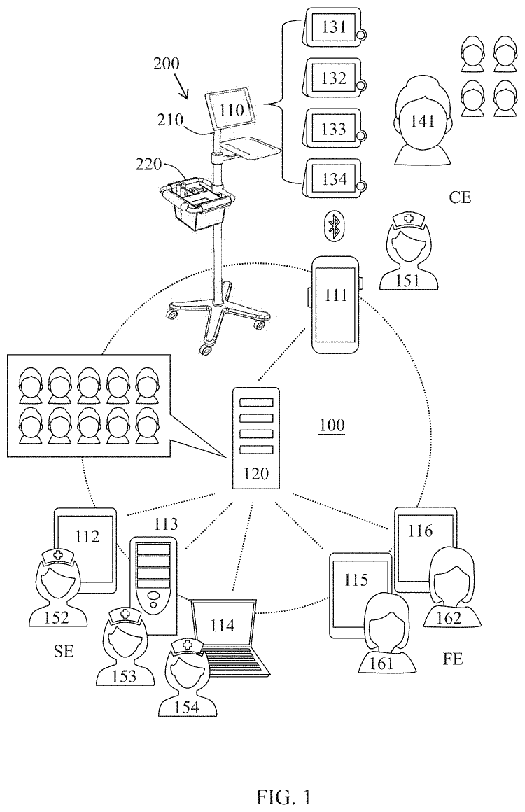

FIG. 1 is a structural schematic view illustrating a nursing information module automation system in accordance with the present invention. The nursing information module automation system 100 provided in the present invention is a cloud-based system composed of a series of related hardware devices which are distributed at a clinical end (CE), a nursing station end (SE), and a family end (FE), a set of distributed intelligent nursing platform software program operating on these related hardware devices, and a nursing information module automation natural language processing model which is built based on a natural language processing (NLP) technology, wherein the nursing information module automation natural language processing model is either incorporated into a nursing information system (NIS) or operating on an intelligent nursing platform of the current embodiment. The clinical end (CE) refers to a periphery centered by a care-receiver 141 who receives a nursing service. In some embodiments, the clinical end (CE) and the nursing station end (SE) are combined and deemed an institutional end where a medical or nursing facility is located.

There is a vary number of user equipment (UE) respectively distributed at the clinical end (CE), the nursing station end (SE), and the family end (FE), including but not limited to: a smartphone 111, tablet devices 110, 112, 115, and 116, a desktop computer 113, and a notebook computer 114. The smartphone 111 and the tablet devices 112, 115, and 116 are all provided with a touch screen unit and are installed with an intelligent nursing platform front-end program, wherein the front-end program refers to an application (APP) or a web page browser. The desktop computer 113 and the notebook computer 114 are all provided with a monitor and input devices such as a mouse and a keyboard. The user equipment 111-116 establishes a communication link with a system server 120 through a built-in wireless radio frequency communication module or wired network, whereby to create a communication connection therebetween. Preferably, the wireless radio frequency communication module includes but not limited to commonly used Wi-Fi, Bluetooth or Bluetooth Low Energy (BLE) module, Sub-1G module, and 4G or 5G mobile communication module. Preferably, the wired network includes but not limited to commonly used Ethernet. Preferably, the communication link between each of the user equipment 111-116 and the system server 120 is composed of combinations of multiple wired or wireless communication links.

The system server 120 is installed with an intelligent nursing platform back-end management program. Through the communication link, the intelligent nursing platform front-end program operated on each of the user equipment 111-116 including APPs or browsers would be capable of accessing the intelligent nursing platform back-end management program on the system server 120, i.e., uploading or downloading date to or from the intelligent nursing platform back-end management program, or receiving control commands from the intelligent nursing platform back-end management program. Preferably, the nursing information module automation system 100 provided in the present invention is built based on a cloud-based technology of Software as a Service (SaaS) and Platform as a Service (PaaS).

A plurality of IoT based nursing equipment with network connecting capability includes but not limited to a network connectable sphygmomanometer 131, a network connectable forehead thermometer 132, a network connectable heart rate monitor 133, and a network connectable oximeter 134, which are provided to measure fundamental vital signs of the care-receiver 141. Preferably, the IoT nursing equipment has a built-in wireless communication module which can be used to establish a wireless LAN (WLAN) or IoT over long distances. Preferably, the protocol applied by the built-in wireless communication module includes but not limited to communication protocols of commonly used Wi-Fi, Bluetooth, Bluetooth Low Energy, or Sub-1G, wherein the Sub-1G module is preferably to be all kinds of radio frequency communication module using ISM bands, of which commonly seen kinds include but not limited to 868 MHz module, 916 MHz module, 926 MHz module, NB-IOT module, Zigbee module, Xbee module, Z-Wave module, LoRa module, etc.

Through the built-in wireless communication module, the IoT nursing equipment establishes a communication connection with a smartphone 111 which is nearby or is also located at the clinical end (CE). In addition, the IoT nursing equipment updates measured vital signs to the intelligent nursing platform front-end program on the smartphone 111 in real-time, and then the updated vital signs will be updated to the intelligent nursing platform back-end management program on the system server 120 in real-time through the intelligent nursing platform front-end program. Furthermore, these measured vital signs are saved in a cloud database indicated by the intelligent nursing platform back-end management program. A caretaker 151 could also update information, which is inputted via the front-end program, to the intelligent nursing platform back-end management program on the system server 120 in real-time through the intelligent nursing platform front-end program.

Preferably, the caretaker (i.e., the healthcare provider) refers to a person who provides medical and health care services, including but not limited to a nurse, a nurse practitioner, a health care worker, a home health aide, and a caretaker. Preferably, the care-receiver (i.e., the healthcare-receiver) is a person who receives nursing or care, including but not limited to a resident, a patient, an elderly, or a disabled.

A smart care work stand 200 could provide a mobile device connecting point 210 at an appropriate height above the ground to fixedly hold the smartphone 111, and could also provide a convenient member 220 to collect and accommodate one or more of the IoT nursing equipment mentioned above in one location, so that the caretaker 151 could easily move and manage the multiple IoT nursing equipment whether it is moving from the nursing station end (SE) to the clinical end (CE) or it is moving between the beds of multiple care-receivers. Heights of the mobile device connecting point 210 and the convenient member 220 above the ground could be adjusted in accordance to the height of the caretaker 151, so that it would be convenient for the caretaker 151 to operate and use the smartphone 111 and the multiple IoT nursing equipment.

Preferably, the smart care work stand 200 could be used as a bedside assistant, a clinical assistant, or could be equipped at a mobile nursing station end. The detailed structure and the involved relevant techniques have been disclosed in Taiwan Patent No. I687209, which is owned by the same patentee, and are protected by said patent, while the patentee has the corresponding patent right.

Through the use of the smart care work stand 200, the caretaker 151 could choose to hold the smartphone 111 in hand, or to fix the smartphone 111 at the mobile device connecting point 210 of the smart care work stand 200. When the smartphone 111 and the IoT nursing equipment have communication connection established therebetween, the readings measured by the IoT nursing equipment from the care-receiver 141 could be uploaded to the intelligent nursing platform back-end management program on the system server 120 in real-time through the intelligent nursing platform front-end program on the smartphone 111, and then the readings would be pushed back to the intelligent nursing platform front-end program on the smartphone 111 at the clinical end (CE) through the intelligent nursing platform back-end management program, or pushed to the intelligent nursing platform front-end program on the user equipment 112-114 at the nursing station end (SE), whereby to display to other caretakers 152-154 synchronously. Or, the readings could be further optionally pushed to the intelligent nursing platform front-end program on the mobile devices 115-116 at the family end (FE), displaying to multiple family members 161-162 synchronously.

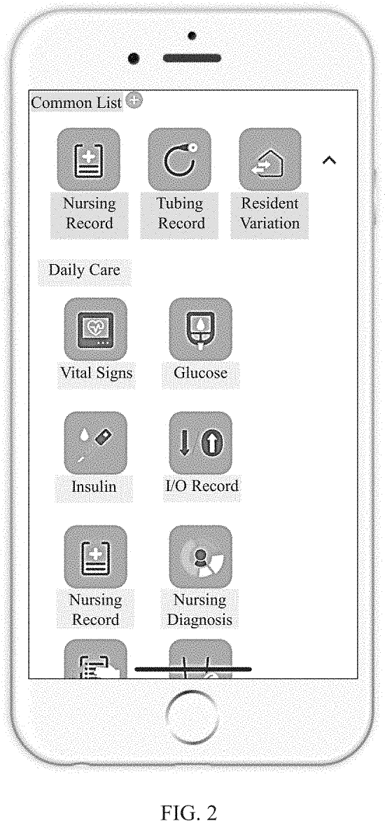

FIG. 2 is a schematic view illustrating the function modules contained in a fully functional intelligent nursing platform provided to caretakers through the front-end program in accordance with the present invention. A fully functional nursing information system or intelligent nursing platform would provide various functional module for caretakers to choose and use, including but not limited to: nursing record module, tubing record module, resident variation module, daily care module, vital signs module, glucose record module, insulin module, I/O record module, nursing diagnosis module, medication plan module, medical record module, fall record module, stress injury module, physical assessment module, physical therapy assessment module, restraint assessment module, pain assessment module, mini nutrition assessment module, fall assessment module, occupational therapy assessment module, nutrition assessment module, interprofessional care module, or examination report module.

However, according to statics, among so many functional modules, the usage rate of commonly used ones such as the nursing record module is higher than 75%. In contrast, the usage rate of less-used modules such as the visit record module is as low as 13.79%. But according to user experience, the use requirement of caretakers (i.e., users) for these modules can be predicted by interpreting the content of the nursing records. For instance, by interpreting the two nursing records listed in the Table 1 below, one could conclude that the nursing focus is an outpatient clinic.

TABLE 1

| Nursing Records | Category | Focus |
|---|---|---|
| Went to the Infectious Disease Department today, | D | outpatient |
| and the doctor has continued to prescribe anti- | | clinic |
| biotics for seven days. Scheduled to return to the | | |
| clinic on 7/14. | | |
| The elder returned to the gastroenterology clinic | D | outpatient |
| in a few days, abdominal ultrasound is normal, | | clinic |
| and there is no hepatitis C. Scheduled to return | | |
| to the clinic on 10/21. | | |

Therefore, the present invention proposes to build a nursing information module automation natural language processing model based on NLP techniques, wherein the model interprets nursing records through a transfer learning algorithm that has been fine-tuned in advance with labeled texts, and the model is incorporated into an intelligent nursing platform to be operated therein. When a caretaker uses the nursing record module to make nursing records, the model would immediately detect and interpret the wordings inputted by the caretaker and predict the nursing focus. According to the predicted focus, the model recommends relevant matching modules to the caretaker so that the operation of the entire nursing information system or the intelligent nursing platform could be automated, enhancing the working efficiency.

Preferably, the nursing information module automation natural language processing model proposed in the present invention is built through implementing a word embedding procedure, an NLP model pre-training procedure, and a fine-tuning procedure, wherein the word embedding procedure roughly includes a step of token embedding, a step of segment embedding, and a step of position embedding, etc. After execution, the word embedding procedure could convert texts into a numerical vector form, which is preferably selected from the group consisting of the one-hot encoding technique, the Word2Vec technique, the Doc2Vec technique, the Glove technique, the FastText technique, the ELMO technique, the GPT technique, and the BERT technique.

The NLP model pre-training procedure roughly includes a masked language modeling (MLM) training and a next sentence prediction (NSP) training. Preferably, it is trained by using an unsupervised learning on an unlabeled corpus. With a self-attention mechanism and a deep bi-directional language model, a large amount of unlabeled corpus could be read through to complete the pre-training of the model. Preferably, the unlabeled corpus includes Wikipedia, BooksCorpus, etc. Preferably, the nursing information module automation natural language processing model provided in the present invention is selected from the ground including but not limited to Transformer model, BERT model, ELMO model, LSTM model, GPT1.0 model, GPT2.0 model, Flair model, StanfordNLP model, and ULMFiT model.

Preferably, the labeled text for fine-tuning undergoes data pre-processing. The data pre-processing is done by collecting a lot of raw data of nursing records of care-receivers in advance, wherein the raw data is, preferably, saved and recorded in a cloud database as digital files, deleting outliers from the raw data, deleting error data, removing incomplete records, and performing field recognition, text recognition, semantics recognition, format conversion, standardization or formatting, whereby to leave correct and reliable data while removing incorrect and missing records. Preferably, the raw data which is used is recorded by using the DART method for recording nursing focuses, which contains subjective and objective data, nursing measures, responses, and nursing instructions, wherein each complete nursing record would have a focus corresponding thereto.

After that, the process of nursing focus labeling will be performed. At this stage, the contents of nursing records are reviewed by professional nurse practitioners, humans, or artificial intelligence to search for the nursing focus corresponding to each segment, and a corresponding label will be added thereto. For instance, say the content of one nursing record is “Due to the expiration of the tube, replaced with a urinary catheter 16fr; resting in bed” and so on, then one could label the nursing focus corresponding to this segment as “urinary catheter replacement”. During the process of labeling, nursing records are used as training texts, while the focuses are the labels thereof.

The final stage is to separate a training dataset from a test dataset, wherein the training dataset includes a validation dataset. The labeled texts are separated into a training dataset and a test dataset in a roughly even manner, which are used to fine-tune the nursing information module automation natural language processing model. The model will learn and interpret nursing records, and will automatically summarize and produce nursing focuses. In the current embodiment, there are more than 160,000 nursing records are collected for training, and the resultant training accuracy exceeds 96%.

Preferably, in the current embodiment, nursing focuses are organized into at least the following 45 categories, but not limited to: life record, hyperthermia, hospitalization, nutritional assessment, notes, itchy skin, poor digestion, abnormal body temperature, bed change, hemodialysis, tube self-extraction, rash, diarrhea, hypertension, return to hospital, pain, tube replacement, edema, constipation, out-of-hospital medication, withdrawal, nutritional assessment, rash, diarrhea, hypertension, return to hospital, pain, tube change, edema, constipation, out-of-hospital medication, hospital discharge, nutritional assessment, stress injury, cough with sputum, vomiting, blood glucose instability, physical assessment, outpatient, general wound, respiratory tract clearance failure, abnormal bowel movements, blood glucose measurement, physical mobility disorder, medical treatment, wound care, hematuria, admission, rehabilitation, accidents: falls, scratch wound, shortness of breath, leave of absence, restraints, potentially hazardous falls, and physical exams.

When the fine-tuning of the nursing information module automation natural language processing model is completed, the nursing information module automation natural language processing model provided in the present invention would be able to predict several possible nursing focuses based on the content of a detected nursing record, and could then display the most likely one or the top 5 to the caretaker after ranking by probabilities. Examples are shown in the Table 2 below, wherein there are ten pairs of nursing records and the corresponding nursing focuses predicted by the model.

TABLE 2

| | | The top 5 nursing focuses |
|---|---|---|
| | Content of nursing records | predicted by the model |
| 1 | Patient has replaced the | [‘tube replacement’, ‘tube self- |
| | bladder stoma due to | extraction’, ‘return to hospital’, |
| | expiration of the tube. | ‘nutritional assessment’, ‘physical |
| | | mobility disorder’] |
| 2 | Patient has not had a bowel | [‘constipation’, ‘diarrhea’, ‘life |
| | movement for 3 days. | record’, ‘return to hospital’, |
| | | ‘wound care’] |
| 3 | Assisting in preparing items | [‘hospitalization’, ‘admission’, |
| | for hospitalization, | ‘return to hospital’, ‘bed change’, |
| | contacting ambulance and | ‘shortness of breath’] |
| | family. | |
| 4 | Hit in the right elbow by the | [‘wound care’, ‘general wound’, |
| | patient self, causing a 0.8 × 0.8 | ‘physical assessment’, ‘scratch |
| | cm wound. Give him a w'd CD | wound’, ‘stress injury’] |
| | gauze to cover the wound. | |
| 5 | Patient returned for dialysis, | [‘hemodialysis’, ‘rehabilitation’, |
| | now has gained 1.7 kb of | ‘physical exams’, ‘blood glucose |
| | weight. Having his meal at | measurement’, ‘potentially |
| | lobby now, with no ill | hazardous pulmonary inhalation’] |
| | feelings. | |
| 6 | Da-ren Pharmacy returns 2 kinds | [‘physician visits’, ‘out-of-hospital |
| | of conventional medicine for 28 | medication’, ‘outpatient’, |
| | days to Ren-an Internal | ‘abnormal bowel movements’, |
| | Medicine Clinic today. | ‘itchy skin’] |
| 7 | Brought back to the hospital by | [‘leave of absence’, ‘return to |
| | his daughter, in a happy mood. | hospital’, ‘life record’, |
| | | ‘admission’, ‘rehabilitation’] |
| 8 | At 19:13 recheck, HR: 59, BP: | [‘hypertension’, ‘return to |
| | 142/57 mmHg. | hospital’, ‘accidents: falls’, ‘blood |
| | | glucose instability’, ‘vomiting’] |
| 9 | Life - TP: 37.6/SPO2: 94, | [‘hyperthermia’, ‘wound care’, |
| | hydrated. | ‘admission’, ‘return to hospital’, |
| | | ‘pain’] |
| 10 | Finished his lunch, with no | [‘anxiety’, ‘poor digestion’, |
| | vomiting or ill feelings. | ‘constipation’, ‘hyperthermia’, |
| | | ‘family visits’] |

Once the nursing focus prediction function has been fully established, a knowledge base of focused modules could be then set up, whereby to create a focus and module matching relationship between nursing focuses and the relevant functional modules. In this way, the model could directly provide corresponding functional modules to the caretaker after the model completes the nursing focus prediction.

Preferably, the matching relationship between nursing focuses and the corresponding functional modules is organized in a way shown in the Table 3 below. If the matching relationship between nursing focuses and functional modules is established, the model could recommend and provide matching functional modules to the caretaker immediately after predicting the nursing focuses, waiting for the caretaker to confirm and perform subsequent operations. Our hope is to provide the caretaker with an information system that is more convenient to use.

TABLE 3

| Functional module | Nursing focus |
|---|---|
| Daily care | stress injury, general wound, wound care, scratch |
| | wound, itchy skin, rash, edema, cough with |
| | sputum, respiratory tract clearance failure, poor |
| | digestion, diarrhea, constipation, vomiting, |
| | abnormal bowel movements, hematuria, shortness |
| | of breath |
| Medication plan | out-of-hospital medication |
| Blood glucose | blood glucose instability, blood glucose |
| record | measurement |
| Resident variation | admission, leave of absence, hospitalization, bed |
| | change, return to hospital, withdrawal |
| Medical record | outpatient, medical treatment |
| Fall record | Accidents: falls |
| Tubing record | tube self-extraction, tube replacement |
| Stress injury | stress injury, general wound, wound care, scratch |
| | wound |
| Physical assessment | physical assessment, life record |
| Physical therapy | physical therapy assessment, rehabilitation |
| assessment | |
| Restraint assessment | restraints |
| Pain assessment | pain |
| Mini-nutritional | nutritional assessment |
| assessment | |
| Fall assessment | potentially hazardous falls |
| Occupational | rehabilitation |
| therapy assessment | |
| Nutritional | nutritional assessment |
| assessment | |
| Interprofessional | notes |
| care | |
| Examination report | physical exams |
| IO | poor digestion, diarrhea, constipation, vomiting, |
| | abnormal bowel movements, hematuria, |
| | hemodialysis |

The nursing record focus prediction, which is performed by the nursing information module automation natural language processing model provided in the present invention, is essentially a multi-class classification problem, wherein the model has to determine what focus a nursing record belongs to, and the prediction result will be converted into corresponding functional modules in the intelligent nursing platform through the focus module knowledge database, by which the modules could be recommended to the caretaker.

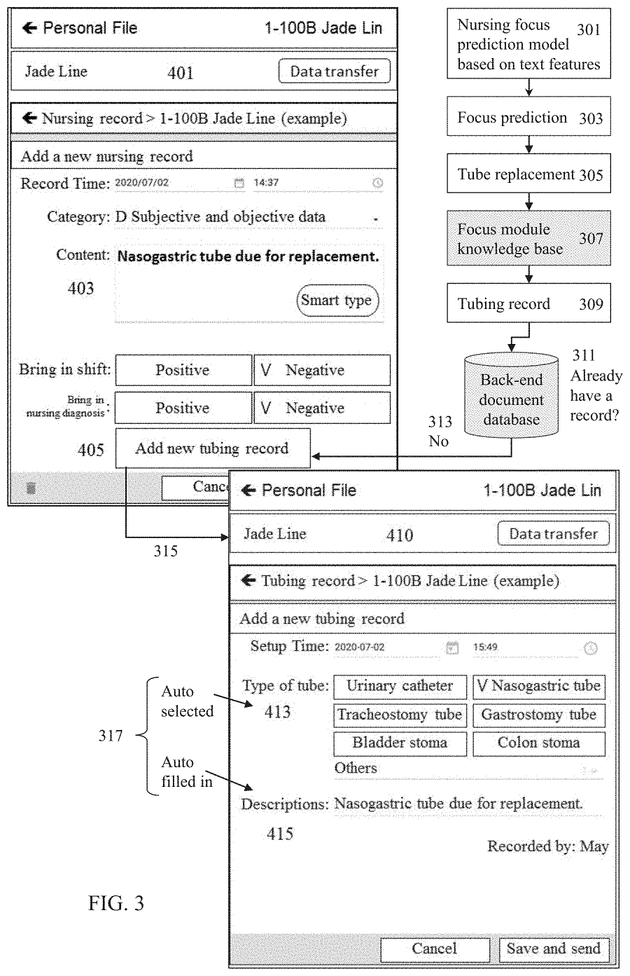

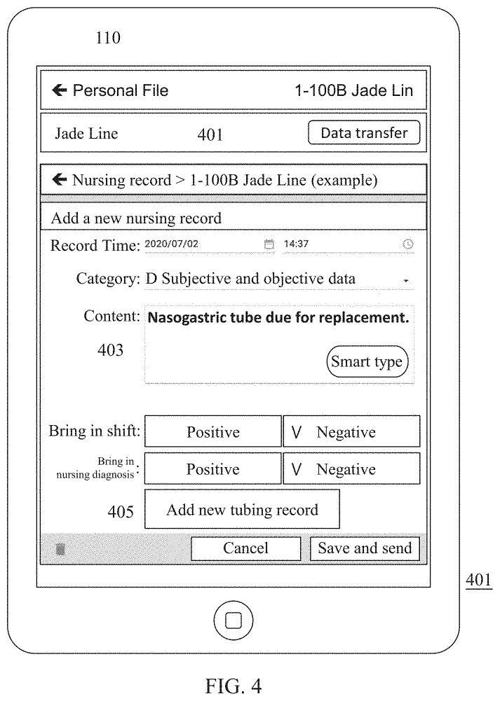

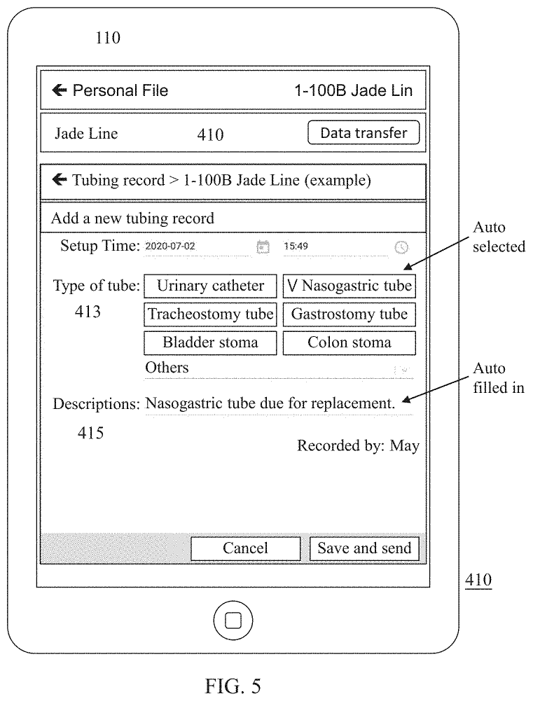

FIG. 3 is a flow chart illustrating operational steps for the nursing information module automation natural language processing model provided in accordance with the present invention. FIG. 4 and FIG. 5 are schematic views illustrating the operations of the nursing information module automation natural language processing model while it is running in a functional module operation interface provided in accordance with the present invention. After the nursing information module automation natural language processing model provided in the present invention is completely built, the operation steps and flowchart of the system are roughly the same as what is disclosed in FIG. 3. As disclosed in FIG. 4 and FIG. 5, the caretaker 151 operates the nursing record module through a front-end program nursing record operation interface 401 to make a nursing record by following the DART method, which is not a limitation of the present invention, whereby the condition of the care-receiver is recorded in the content field 403. In the current embodiment, as shown in FIG. 4, the caretaker 151 notes “Nasogastric tube due for replacement” in the content field 403 as an example.

When the system detects that the caretaker 151 inputs a nursing record into the nursing record module, the system immediately performs the nursing information module automation natural language processing model, wherein, as shown in Step 301, the model immediately detects and interprets the semantics of the text inputted by the caretaker 151. In the current embodiment, the predicted nursing focus is “tube replacement”, which is an example and shown in Step 305. Once the nursing focus prediction is completed, the model immediately accesses the focus module knowledge database, as shown in Step 307. According to the predicted nursing focus, the model finds the corresponding functional module in the focus module knowledge database, and uses it as the recommended module. In the current embodiment, the recommended module found by the model is the tubing record module, as shown in Step 309.

After the recommended module is determined, the model further accesses a back-end document database of the intelligent nursing platform, as shown in Step 311, wherein the database includes but not limited to MongoDB. The model searches to see if the care-receiver already has a related record in the recommended module. If there is one, the model retrieves the related record, and displays it on the tablet device 110 for the caretaker 151; otherwise, the model generates an “Add a new tubing record” button 405 on the front-end program nursing record operation interface 401, as shown in Step 313, whereby to recommend the tubing record module to the caretaker 151.

After the caretaker 151 clicks the “Add a new tubing record” button 405, the system enters the tubing record module 410 as shown in FIG. 5 and Step 315. The model implements NLP-related techniques, including but not limited to semantics extraction technology. Semantics extraction technology could identify main vocabularies and common vocabularies based on the fields in relevant modules and the record content histories, and could build a keyword dictionary, so that relevant information could be extracted from the nursing record noted by the caretaker 151. Furthermore, the model would automatically select an option in an appropriate field in the tubing record module. For example, the model automatically selects the “Nasogastric tube” option in the “Type of tube” field 413 in the current embodiment. In addition, the model also automatically fills a note “Nasogastric tube due for replacement.” in the “Descriptions” field 415, as shown in Step 317.

Based on the test results, the model provided in the present invention could provide a 96% accuracy for the top 5 nursing focus predictions. In other words, when the model recommends a module list to the caretaker 151, it could effectively provide correct module choices to the user. With natural language technology which could extract information for bringing into the fields, the model could save time for caregivers and ensure data integrity.

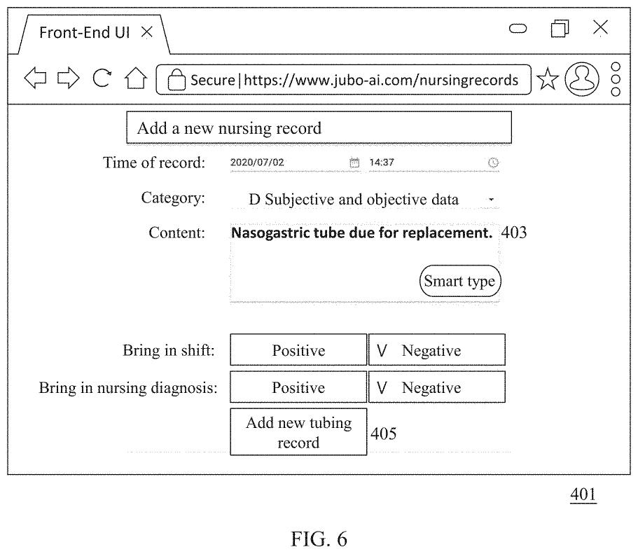

FIG. 6 is a schematic view illustrating how the nursing information module automation system operates in a webpage browser provided in accordance with the present invention. The nursing information module automation system 100 of the present invention is built based on Software as a Service (SaaS) and Platform as a Service (PaaS), and could provide the nursing information system or the intelligent nursing platform to the caretaker 154 on a front-end presentation layer through a browser which runs on a notebook computer 114 or a tablet device 115, wherein the type of the device that has the browser is not a limitation of the present invention. Preferably, the browser runs on the user equipment, which includes but not limited to: smartphone 111, tablet devices 110, 112, 115, and 116, desktop computer 113, notebook computer 114, etc.

When connected to the Internet, the caretaker 154 could launch the browser on the tablet device 110 owned by the caretaker 154 and could enter a correct URL in the address bar, as disclosed in FIG. 6. In this way, the caretaker 154 would be able to access the nursing information system or the intelligent nursing platform through the browser, and could activate the nursing information module automation natural language processing model provided in the present invention. As shown in FIG. 6, the caretaker 151 enters “Nasogastric tube due for replacement” into the content field 403 of the nursing record module. The system determines that the care-receiver has no related record in the back-end document database, and therefore the model generates a “Add a new tubing record” button 405 in the nursing record operation interface 401 of the nursing record module.

The nursing information module automation natural language processing model provided in the present invention has two main parts, which are the nursing focus prediction and the semantics extraction. First, the model performs the “focus prediction” and determines what nursing focus belongs to the nursing record. Furthermore, a multiple classification task for the text is performed, and then a recommended module list is obtained through the matching in the focus module knowledge database. After that, the determined modules are recommended. In addition, before the step of recommending modules, the model also checks if it is necessary to go into that step based on the previous records of the user. If the module has an unfilled field, the “semantic extraction” model would be used to extract the content of the nursing record to bring into the selected module. The nursing information module automation natural language processing model provided in the present invention at least has three effects, including: automatically creating a nursing focus, providing a caring module recommendation system, and automatically filling out the modules.

The model provided in the present invention could interpret nursing records and recommend nursing modules, so that the caretaker could record the nursing process according to the condition of the care-receiver. The model could assist the caretaker in searching for relevant modules and recommend them to the caretaker, increasing the working efficiency. Therefore, the model provided in the present invention could assist nurse practitioners in medical facilities, and could search unfilled modules related to the content of a nursing record with the aid of semantics extraction to increase the efficiency in making records. In addition, the model could assist caretakers in medical facilities. If there are related contents in daily care modules in nursing records, the related contents would be brought in as well. Furthermore, the model could assist professional doctors with professional modules such as nutrition and occupational/physical therapy assessments. The recommendations made by the model could also be provided with a reminding mechanism according to the nursing focus predictions. We hope to provide a more comprehensive and more convenient system for all kinds of caregivers.

The model proposed in the present invention could assist nurse practitioners in filling out nursing records by the module recommendation system to fill in the related professional modules quickly, so as to improve the efficiency of filling in the modules and ensure the data integrity of the professional modules. Furthermore, the model could remind a nurse practitioner whether there are modules that should be filled in but not filled in, and to retrieve the text information and bring it into the related fields of the selected modules automatically.

There are further embodiments provided as follows.

Embodiment 1: a nursing information module automation method, includes step of: detecting a nursing record inputted by a user; transmitting the nursing record to a nursing information module automation natural language processing model to perform a focus prediction, so as to automatically predict at least one nursing focus based on the nursing record; and recommending at least one recommended module for the user automatically in accordance with the at least one nursing focus by the nursing information module automation natural language processing model.

Embodiment 2: the nursing information module automation method as described in Embodiment 1, further includes one of following steps: accessing a focus module knowledge database by the nursing information module automation natural language processing model, to recommend the at least one recommended module for the user, in accordance with a focus and module matching relationship in the focus module knowledge database based on the at least one nursing focus; and implementing a semantics extraction technology by the nursing information module automation natural language processing model, to import the nursing record inputted by the user into the at least one recommended module automatically.

Embodiment 3: the nursing information module automation method as described in Embodiment 1, further includes one of following steps: implementing a word embedding technology for an unlabeled corpus and a labeled text to transform a word into a numerical status; pre-training the nursing information module automation natural language processing model based on the unlabeled corpus by using an unsupervised machine learning scheme; fine-tuning the nursing information module automation natural language processing model based on the labeled text by using a supervised machine learning scheme.

Embodiment 4: the nursing information module automation method as described in Embodiment 3, the word embedding technology is selected from one of a One-hot encoding technology, a Word2Vec technology, a Doc2Vec technology, a Glove technology, a FastText technology, an ELMO technology, a GPT technology, a BERT technology and a combination thereof.

Embodiment 5: the nursing information module automation method as described in Embodiment 3, the nursing information module automation natural language processing model is selected from one of a Transformer model, a BERT model, an ELMO model, a LSTM model, a GPT1.0 model, a GPT2.0 model, a Flair model, a StanfordNLP model, a ULMFiT model and a combination thereof.

Embodiment 6: a nursing information module automation system, includes steps of: a system server configured to install an intelligent nursing platform including the nursing information module automation natural language processing model; and a user equipment communicatively connected with the and system server and configured to install a frontend interface program to detect a nursing record inputted by a user, wherein the user equipment transmits the nursing record to the nursing information module automation natural language processing model to perform a focus prediction, so as to automatically predict at least one nursing focus based on the nursing record, and generate at least one recommended module based on the predicated at least one nursing focus, and displays the at least one recommended module for the user via the frontend interface program.

Embodiment 7: the nursing information module automation system as described in Embodiment 6, the intelligent nursing platform further includes one of a nursing record module, a tubing record module, a resident variation module, a daily care module, and the at least one recommended module.

Embodiment 8: the nursing information module automation system as described in Embodiment 7, the user inputs the nursing record into the intelligent nursing platform by operating the nursing record module.

Embodiment 9: the nursing information module automation system as described in Embodiment 7, by implementing a semantics extraction technology, the nursing information module automation natural language processing model imports the nursing record inputted by the user by operating the nursing record module into the at least one recommended module automatically.

Embodiment 10: the nursing information module automation system as described in Embodiment 6, the user equipment is selected from one of a mobile device, a smart phone, a tablet device, a desktop computer and a notebook computer.

While the disclosure has been described in terms of what are presently considered to be the most practical and preferred embodiments, it is to be understood that the disclosure need not be limited to the disclosed embodiments. On the contrary, it is intended to cover various modifications and similar arrangements included within the spirit and scope of the appended claims, which are to be accorded with the broadest interpretation so as to encompass all such modifications and similar structures. Therefore, the above description and illustration should not be taken as limiting the scope of the present disclosure which is defined by the appended claims.

What is claimed is:

1. A nursing information module automation method, comprising:
detecting a nursing record inputted by a user;
transmitting the nursing record to a nursing information module automation natural language processing model to perform a focus prediction, so as to automatically predict at least one nursing focus based on the nursing record; and
recommending at least one recommended module for the user automatically in accordance with the at least one nursing focus by the nursing information module automation natural language processing model.

2. The nursing information module automation method as claimed in claim 1, further comprising one of following steps:
accessing a focus module knowledge database by the nursing information module automation natural language processing model, to recommend the at least one recommended module for the user, in accordance with a focus and module matching relationship in the focus module knowledge database based on the at least one nursing focus; and
implementing a semantics extraction technology by the nursing information module automation natural language processing model, to import the nursing record inputted by the user into the at least one recommended module automatically.

3. The nursing information module automation method as claimed in claim 1, further comprising one of following steps:
implementing a word embedding technology for an unlabeled corpus and a labeled text to transform a word into a numerical status;
pre-training the nursing information module automation natural language processing model based on the unlabeled corpus by using an unsupervised machine learning scheme;
fine-tuning the nursing information module automation natural language processing model based on the labeled text by using a supervised machine learning scheme.

4. The nursing information module automation method as claimed in claim 3, wherein the word embedding technology is selected from one of a One-hot encoding technology, a Word2Vec technology, a Doc2Vec technology, a Glove technology, a FastText technology, an ELMO technology, a GPT technology, a BERT technology and a combination thereof.

5. The nursing information module automation method as claimed in claim 3, wherein the nursing information module automation natural language processing model is selected from one of a Transformer model, a BERT model, an ELMO model, a LSTM model, a GPT1.0 model, a GPT2.0 model, a Flair model, a StanfordNLP model, a ULMFiT model and a combination thereof.

6. A nursing information module automation system, comprising:
a system server configured to install an intelligent nursing platform including the nursing information module automation natural language processing model; and
a user equipment communicatively connected with the and system server and configured to install a frontend interface program to detect a nursing record inputted by a user,
wherein the user equipment transmits the nursing record to the nursing information module automation natural language processing model to perform a focus prediction, so as to automatically predict at least one nursing focus based on the nursing record, and generate at least one recommended module based on the predicated at least one nursing focus, and displays the at least one recommended module for the user via the frontend interface program.

7. The nursing information module automation system as claimed in claim 6, wherein the intelligent nursing platform further comprises one of a nursing record module, a tubing record module, a resident variation module, a daily care module, and the at least one recommended module.

8. The nursing information module automation system as claimed in claim 7, wherein the user inputs the nursing record into the intelligent nursing platform by operating the nursing record module.

9. The nursing information module automation system as claimed in claim 7, wherein, by implementing a semantics extraction technology, the nursing information module automation natural language processing model imports the nursing record inputted by the user by operating the nursing record module into the at least one recommended module automatically.

10. The nursing information module automation system as claimed in claim 6, wherein the user equipment is selected from one of a mobile device, a smart phone, a tablet device, a desktop computer and a notebook computer.
